SEQUENCE LISTING (1 sequence)

SEQ ID NO: 1 - DNA, 52 bp, synthetic construct
  source 1..52: mol_type = other DNA
ggacgacaccgctgcgatacggtgatacaattgtaccgcactggattgtcgt

(19) United States
(12) Patent Application Publication
(10) Pub. No.: US 2026/0345071 A1
(43) Pub. Date: Nov. 5, 2026

(54) BIOSENSOR SYSTEM FOR DRUG EFFICACY MONITORING

(71) Applicant: University of Cincinnati, Cincinnati, OH (US)

(72) Inventors: Jason Charles Heikenfeld, Cincinnati, OH (US); Zachary Lee Watkins, Cincinnati, OH (US)

(21) Appl. No.: 19/102,459

(22) PCT Filed: Aug. 10, 2023
(86) PCT No.: PCT/US2023/030025

§ 371 Date: Feb. 10, 2025

Related U.S. Application Data
(60) Provisional application No. 63/396,811, filed on Aug. 10, 2022.
(60) Provisional application No. 63/412,044, filed on Sep. 30, 2022.

(51) Int. Cl. A61B 5/00; A61B 5/02; A61B 5/0205; A61B 5/145; A61B 5/1473; A61B 5/1495; A61B 5/346

(52) U.S. Cl. CPC A61B 5/4848; A61B 5/0205; A61B 5/14735; A61B 5/1495; A61B 5/4833; A61B 5/02028; A61B 5/14514; A61B 5/346

(57) ABSTRACT

A novel system for monitoring the efficacy of a drug administered to a subject is disclosed. The system includes at least one aptamer sensor capable of providing a first measurement of the drug, and a second device or sensor capable of providing a second measurement regarding the efficacy or impact of the drug.

### CROSS-REFERENCE TO RELATED APPLICATIONS

This application claims priority to, and the benefit of the filing date of, U.S. Provisional Application No. 63/396,811 filed Aug. 10, 2022, and U.S. Provisional Application No. 63/412,044 filed Sep. 30, 2022, the disclosures of which are incorporated by reference herein in their entirety.

### FIELD OF THE INVENTION

This invention relates generally to aptamer sensors.

### BACKGROUND OF THE INVENTION

This section is intended to introduce the reader to various aspects of the art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Continuous monitoring technology for multiple analytes finally appears to be close to commercial reality after two decades of intensive pursuit to move beyond just measuring glucose. The first in-vivo animal and human demonstrations are now appearing in the literature for dermal interstitial fluid-based sensing of drug concentrations using sensors such as aptamer sensors. However, for many applications, such as blood thinner monitoring, just measuring the drug itself is not the measure that is most valuable to doctors and patients. The goal is to measure the effect of the drug. Thus, a significant need exists for improved devices and methods that improve the blood-correlation of protein measurements in interstitial fluid (ISF), including simple single-data point readings, multiple data point readings, or even continuous readings.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

In an embodiment of the invention, a novel system for monitoring the efficacy of a drug administered to a subject. The system includes at least one aptamer sensor capable of providing a first measurement of the drug, and a second device or sensor capable of providing a second measurement regarding the efficacy or impact of the drug. In one embodiment, the second measurement is selected from the group consisting of a point of care test, a lab assay, a heart rate measurement and a blood pressure measurement. In another embodiment, the first measurement provides a compliance score for taking the drug. In one embodiment, the first measurement provides a therapeutic window score for the drug.

In another embodiment, the first measurement is continuous and the second measurement is intermittent. In one embodiment, the second measurement is used to calibrate the continuous first measurement into a continuous predictive measure of the intermittent second measurements.

In another embodiment, the drug is an anticoagulant and the second measurement is coagulation time. In one embodiment, the coagulation time measurement occurs from about an hour to two days after the drug was administered to the subject.

In another embodiment, the drug is predinisone and the second measurement is a level of C-reactive protein or IL-6. In one embodiment, the system measures a drug and its effect, and further, the effect cannot be measured with another aptamer sensor. In another embodiment, the drug is an antibiotic and the effect is bacterial killing kinetics of the antibiotic afforded by area under a concentration curve. In one embodiment, the drug is an antibiotic and the effect is time spent above a minimal inhibitory concentration for a given bacterial strain. In another embodiment, the drug is digoxin, and the effect is change in left ventricular ejection fraction time. In one embodiment, the drug is theophylline and the effect is change in forced expiratory volume. In another embodiment, the drug is an antiarrhythmic drug and the effect is change in electrocardiogram features. In one embodiment, the second measurement of the system of the present invention includes a time-stamp and the time-stamp is aligned with time-stamps of the first measure. In another embodiment, the second measurement includes a population data set taken from multiple patients taking the drug.

Another aspect of the present invention involves a method of reporting the pharmacodynamics of a drug in a patient. The method involves in-vitro calibrating a sensor before use by the patient to create a calibration data set. Then, applying the sensor to the patient and measuring raw output of the sensor over a plurality of time points. Next, using the calibration data set to turn the raw output of the sensor into a quantitative measure of drug concentration in the patient. Then, taking a measurement of a pharmacodynamic effect of the drug on the patient using a point-of-care or laboratory measurement, and assigning the time of measurement of the pharmacodynamic effect to a time stamp. Next, using the measure of pharmacodynamic effect to calibrate the raw output of the sensor or the quantitative measure of the drug concentration at the time of the time stamp into a quantitative measure of pharmacodynamic effect. Finally, using the measure of pharmacodynamic effect to provide a plurality of predictive quantitative pharmacodynamic effect measures over time.

In one embodiment, the drug is an antibiotic and the pharmacodynamic effect is bacterial killing kinetics of the antibiotic afforded by area under a concentration curve. In another embodiment, the drug is an antibiotic and the pharmacodynamic effect is time spent above a minimal inhibitory concentration for a given bacterial strain. In one embodiment, the drug is digoxin and the pharmacodynamic effect is change in left ventricular ejection fraction time. In another embodiment, the drug is theophylline and the pharmacodynamic effect is change in forced expiratory volume. In one embodiment, the drug is an antiarrhythmic drug and the pharmacodynamic effect is change in electrocardiogram features.

Another aspect of the present invention involves a method for providing continuous estimates of a patient's pharmacodynamic response to a drug. The method involves having a patient wear an electrochemical aptamer sensing device, wherein the device provides a continuous or semi-continuous stream of raw current data as output from the sensing device. Then, using the raw current data in combination with calibration parameters to calculate continuous or semi-continuous estimates of drug concentrations in the patient, wherein the calibration parameters are selected from the group consisting of batch-calibrated, factory-calibrated, “calibration-free” parameters, and combinations thereof. Next, using the continuous or semi-continuous estimates of drug concentrations in the patient to determine a pharmacokinetic profile for the patient's current dosing regimen of the drug.

In one embodiment, the method also involves correcting or updating the estimates of drug concentrations by using another biosensing device that can periodically provide discrete measures of drug concentrations. In one embodiment, the drug is an anticoagulant. In another embodiment, the drug is rivaroxaban.

### BRIEF DESCRIPTION OF THE DRAWINGS

The objects and advantages of the present invention will be further appreciated in light of the following detailed descriptions and drawings in which:

FIG. 1 is a schematic view of an exemplary sensing device in accordance with an embodiment of the invention.

FIG. 2 is a schematic of a conventional aptamer sensor device.

FIG. 3 is a graph of example human data showing how tightly blood thinner can correlate with prothrombin time, which is a measure like international normalized ratio (INR).

FIG. 4 is a graph showing human pharmacokinetic data for rivaroxaban. The data is for rivaroxaban pharmacokinetics for single 20 mg oral dose (Meuck 2014).

FIG. 5 is a graph showing performance of a rivaroxaban sensor at 33 °C in human serum, which is a proxy for interstitial fluid.

FIG. 6 is an image showing a flow chart concerning pharmacokinetics.

### DEFINITIONS

As used herein, the term “about,” when referring to a value or to an amount of mass, weight, time, volume, pH, size, concentration, or percentage, is meant to encompass variations of, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods and operate the disclosed devices.

As used herein, the term “aptamer” means a molecule that undergoes a conformation change as an analyte binds to the molecule, and which satisfies the general operating principles of the sensing methods and devices as described herein. Such molecules include, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function. Typically, aptamers used in electrochemical sensors are tagged with a redox molecule such as methylene blue.

The devices and methods described herein encompass the use of sensors. A sensor, as used herein, is a device that is capable of measuring the concentration of a target analyte in solution. As used herein, an “analyte” may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of the cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

As used herein, the term “calibration-free” refers to the exploitation of the square wave frequency dependence of electrochemical aptamer sensors, or any other electrochemical means of exploiting differences in electron transfer kinetics as analyte binds to aptamer sensor elements, in order to obtain a ratiometric response that accounts for sensor-to-senor variation and obviates the need for factory calibration of a given sensor.

As used herein, the term “continuous sensing” may be satisfied by a device recording a plurality of readings over a period of time during which the sensing occurs. Thus, even a point-of-care testing device which provides a single data point can be considered a continuous sensing device if, for example, the test has a 15 minute duration, and the testing device operates by taking multiple data points over 15 minutes and averaging them to provide a single data measure.

As used herein, the term “electrode” may apply to any material that is electrically conductive such as gold, platinum, nickel, silicon, conductive liquid infused materials such as ionic liquids, PEDOT: PSS, conductive oxides, carbon, boron-doped diamond, nanotubes or nanowire meshes, or other suitable electrically conducting materials.

As used herein, the term “redox tag” or “redox molecule” means any species such as small or large molecules with a redox active portion that, when brought adjacent to an electrode, can reversibly transfer at least one electron with the electrode. Redox tag or molecule examples include, but are not limited to, methylene blue, ferrocene, quinones, or other suitable species that satisfy the definition of a redox tag or molecule. In some cases, a redox tag or molecule is referred to as a redox mediator. Redox tags or molecules may also exchange electrons with other redox tags or molecules.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “large diluted analyte” means any solute interstitial fluid (ISF) which has at least 10% dilution at some but not all times compared to its concentration in blood due to size-selective partial rejection of the analyte as it attempts to passively transport from blood into ISF.

### DETAILED DESCRIPTION OF THE INVENTION

One or more specific embodiments of the present invention are described below. In an effort to provide a concise description of these embodiments, all features of an actual implementation may not necessarily be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the present invention show sensors as simple individual elements. It is understood that many sensors require two or more electrodes, reference electrodes, or additional supporting technology or features that, for purposes of clarity, are not necessarily described herein. Sensors may measure one or more characteristics of an analyte. Sensors are typically electrical in nature, but may also include optical, chemical, mechanical, or other known sensing mechanisms. Sensors can be in duplicate, triplicate, or more, to provide improved data and readings. Sensors may provide continuous or discrete data and/or readings. Certain embodiments of the present invention may show certain sub-components of sensing devices, but may omit additional sub-components inherent in the device in various applications that are known, e.g., a battery, antenna, or adhesive. These omissions may be for purposes of brevity, and to focus on certain inventive aspects of the disclosed embodiments of the present invention. All ranges of parameters disclosed herein include the endpoints of the ranges.

The present invention involves a novel system for monitoring the efficacy of a drug administered to a subject. The system includes at least one aptamer sensor capable of providing a first measurement of the drug, and a second device or sensor capable of providing a second measurement regarding the efficacy or impact of the drug.

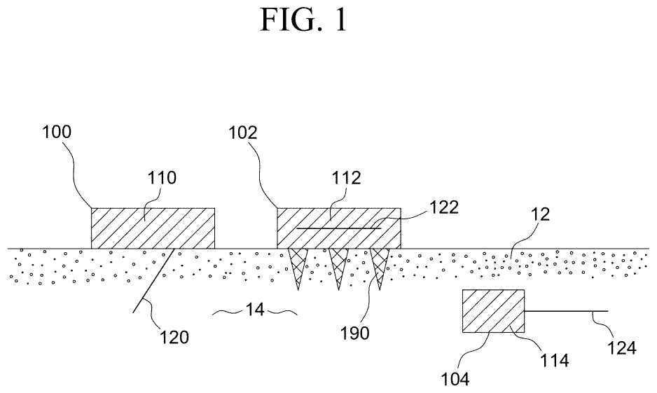

With reference to FIG. 1, in embodiments of the present invention a first device 100, a second device 102 or a third device 104 are configured to measure at least one analyte in the body in interstitial fluid or blood, and are configured on skin 12 (for first device 100 and second device 102) or implanted in the body (for third device 104). Each device has a housing (first housing 110, second housing 112, third housing 114), which may contain electronics and other required components for a sensing device. Each device has a sensor (first sensor 120, second sensor 122, third sensor 124). In the case of first sensor 120, the sensor is inserted into the skin 12. In the case of second sensor 122, the sensor 122 is coupled to biofluid 14 in the skin via porous or hollow microneedles 190. Sensors may be electrochemical or optical based sensors such as aptamers or other affinity-based sensors. The first device 100 may include a sensor for a drug and may additionally include sensors for glucose and or biomarkers related to the drug.

Some embodiments of the present invention use electrochemical aptamer sensors. An electrochemical aptamer sensor may be constructed on a gold wire, a gold-coated needle/microneedle, or on a flexible substrate in the form of a thin strip with thin-film gold deposited on said substrate by one or more of the following methods: physical vapor deposition, electrodeposition, or electroless chemical deposition. Any other suitable transducing electrode material beyond gold may be used on a suitable substrate that can be feasibly inserted into the skin for intradermal monitoring. For sensors prepared on gold or gold-coated substrates, the gold surface may be enhanced prior to functionalization in order to improve sensor performance. This may include physical processes that result in nano structuring of the gold.

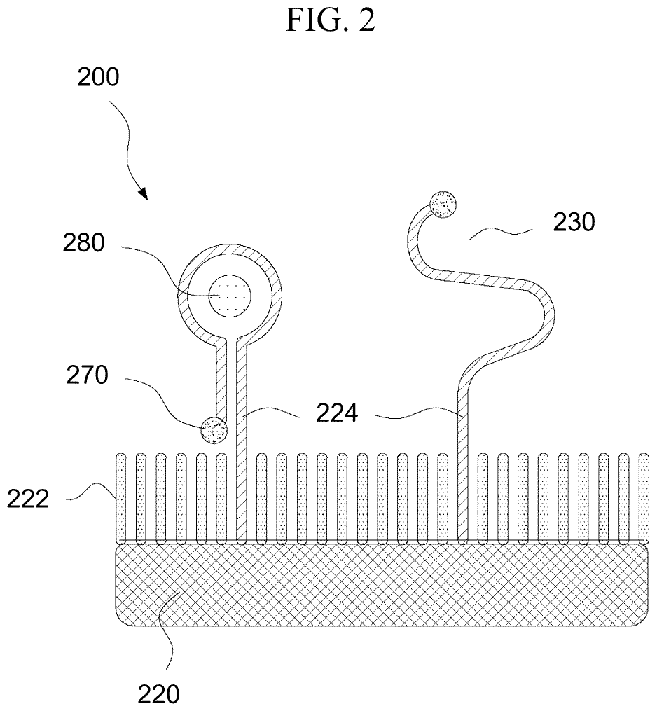

With reference to FIG. 2, a conventional aptamer sensor device 200 as placed initially in a sample fluid 230, such as interstitial fluid, is shown. The sensor comprises: at least one working electrode 220 such as gold, carbon, or other suitable electrode material; at least one blocking layer 222 of a plurality of molecules such as mercaptohexanol or hexanethiol that are thiol bonded to the electrode, or a plurality of natural solutes in blood that can act as a blocking layer, or other suitable molecules depending on application and on the choice of electrode 220 material; at least one aptamer 224 that is responsive to binding to an analyte 280 and which contains a redox tag 270 such as methylene blue. In the generic example taught for FIG. 2, the aptamer 224 is a simple stem loop (hairpin) aptamer where analyte 280 binding causes the stem loop to form and the redox current measured from the redox tag 270 to increase, as measured using square wave voltammetry or other suitable technique. In absence of analyte 280 binding to the aptamer 224 the stem loop is broken and the redox current would decrease. Thus a measurement of electrical current can be used to interpret changes in concentration of the analyte 280.

Once functionalized, an electrochemical aptamer sensor can be tested in vitro to assist with the calibration of sensor readout to the concentration of analyte that is being measured. Briefly, sensors may be tested in a biological fluid that simulates an in vivo testing environment in order to achieve more robust calibration and thus such a test may be carried out in a fluid such as bovine serum at temperatures reflective of those seen in vivo (e.g., 33 °C for sensors placed in the dermis). Sensors are then challenged with increasing concentrations of analyte in that particular biofluid and the sensor response is recorded with an appropriate electrochemical technique, such as square wave voltammetry. Electrochemical parameters may be modulated during these tests in order to determine the most optimal operational values, which may include, for example, modulation of square wave voltammetry frequency, increment, or amplitude. The electrochemical response (e.g., increase in current) at these different parameters is related to the concentration of target analyte added in order to calibrate the sensor. For example, the % increase in current from baseline (i.e., % signal gain) is typically calibrated to the concentration of analyte that is being measured.

### Drug Measurement

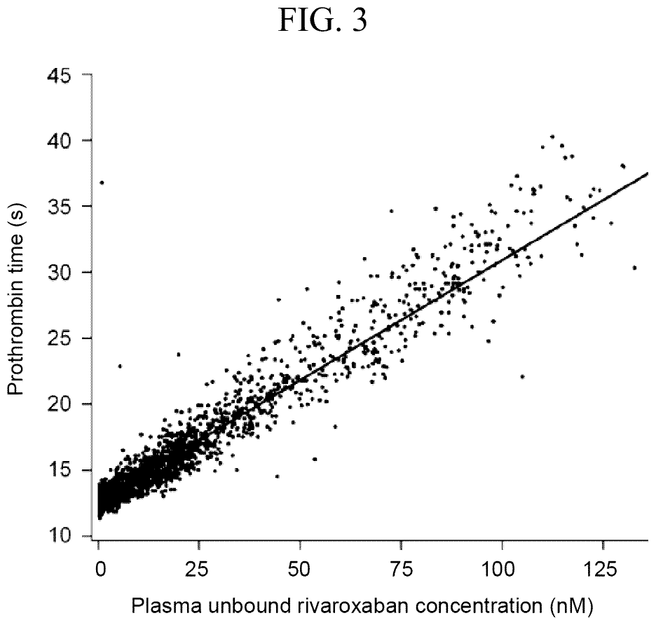

In many cases where drugs are administered to a patient, the drug might be continuously measurable. This has been demonstrated in numerous in-vivo studies for electrochemical aptamer-based sensors. However, measuring the drugs impact is currently done via point-of-care or lab-based tests such as blood analysis. Importantly, these same measures of impact are not easily imported onto a continuous sensing device. For example, as illustrated in FIG. 3, the blood thinner (anticoagulant) drug rivaroxaban has a strong correlation with clotting or coagulation time (Prothrombin time, INR or international normalized ratio) of r~0.9. In an embodiment of the present invention, a system consisting of a first device 100, a second device 102, or a third device 104 may provide a first measure of a drug, and a second device or sensor provides a second measure of efficacy or impact of the drug. Examples of such a second device or sensor include a Coagucheck XS System by Roche or similar system, which demonstrates excellent correlation (97%) with traditional laboratory methods for measuring INR. The time stamp of the continuous first measures and intermittent second measures is measured or known, which can then be used to calibrate the continuous first measure into a continuous predictive measure of the second measure. In one embodiment, INR is measured hours or days after dosing of a drug such as rivaroxaban to achieve this continuous predictive measure of INR because rivaroxaban has a strong correlation of drug concentration to INR. This can be also applied to other blood thinner drugs, such as apixiban and warfarin, where measurement of coagulation time may also be warranted. The system of the present invention, which enables portable or at-home data regarding the efficacy or impact of a drug, could be important for patients and their doctors. For example, the data provided by the present invention may improve compliance of diabetic patients using medications to control their blood glucose levels, resulting better health results for this at-risk group. For example, currently about 5% of diabetics are on warfarin. About 23% of heart valve recipients are diabetics and 27% of them have complications such as clotting, strokes and bleeding. Better awareness of the effectiveness of their diabetes medications may reduce these issues.

A second embodiment of the present invention involves prednisone and C-reactive protein, because C-reactive protein is very large (>120 kDa) and therefore could be difficult to reliably measure in dermal interstitial fluid by devices 100, 102, or 104. Other markers of inflammation may also be used, such as IL1-IL6, CD4+ helper T cells, cyclooxygenases, etc. In another embodiment, an estimate of an endogenous biomarker that is modulated in response to the drug can be used to guide therapy. Examples include immunosuppressant drugs such prednisone, methotrexate, tacrolimus, azathioprine, which for example would lower concentrations of C-reactive protein, IL-6 or other biomarkers. In one embodiment, the drug docetaxel is used as a measurement for prostate cancer, along with an estimate of an endogenous marker that is indicative of response to therapy, such as prostate specific antigen (i.e., PSA).

Another embodiment of the present invention involves the measurement of a drug and its effect when the effect cannot be measured in a routine blood test analysis or point of care test. One example of this embodiment is the measurement of an antibiotic, such as vancomycin, and the estimated bacterial killing kinetics afforded by the area under the concentration curve, or the time spent above the minimal inhibitory concentration for a given bacterial strain. Another example of this embodiment involves the measurement of digoxin concentrations and estimates of the resulting inotropic effects of the drug, such as left ventricular ejection fraction time, which has been shown to be strongly correlated to the drug concentration (r~0.91). One embodiment of the present invention pertains to the measurement of theophylline concentrations and the resulting changes in lung function tests in asthma patients, such as forced expiratory volume (FEV₁). Another embodiment involves antiarrhythmic drugs and the resulting changes in specific electrocardiogram features.

In one embodiment of the present invention, the device and/or system (including software) is pre-programmed to provide a meaningful value even without calibration. For example, a sensor for rivaroxaban can be factory calibrated, and the population linear dependence on INR is strong enough such that INR can be reported to a patient without calibration with a lab assay at a doctors office or with a Coagucheck system at home.

Another embodiment is the measurement of a drug and its effect when the effect is not the intended therapeutic action/pharmacodynamic effect of the drug. Instead, the present invention monitors a particular side effect that is clinically relevant to the drug and the management of its administration. An example of this embodiment would be the measurement of a chemotherapeutic drug such as, for example, cisplatin, carmustine, 5-fluorouracil, or 6-mercaptopurine. The estimate of the immunosuppressant effect of the particular drug that would be typically measured with a complete blood count via a blood draw. In one embodiment, the drug doxorubicin is measured and used in conjunction with the estimate of other endogenous biomarkers, such as cardiac troponins, cardiac CKs, BNP and NT-proBNP, that are indicative of the drugs cardiotoxicity.

With reference to the above-described embodiments of the present invention, the present invention more broadly provides: A method of reporting the pharmacodynamics of a drug in a patient. In one embodiment, the method involves:
a. in-vitro calibrating the sensor before use by the patient to create a calibration data set;
b. applying the sensor to a patient and measuring the raw output of the sensor over a plurality of time points;
c. using the calibration data set to turn the raw output of the sensor into a quantitative measure of drug concentration in the patient;
d. taking a measurement of the pharmacodynamic effect of the drug on the patient using a point-of-care or laboratory measurement, and assigning the time of measurement of the pharmacodynamic effect to a time stamp;
e. using the measure of pharmacodynamic effect to calibrate the raw output of the sensor or the quantitative measure of the drug concentration at the time of the time stamp into a quantitative measure of pharmacodynamic effect;
f. using the measure of pharmacodynamic effect to provide a plurality of predictive quantitative pharmacodynamic effect measures over time.

With further reference to embodiments of the present invention, the second measure may be a point of care test, lab assay, or other measure of drug impact or efficacy such as heart rate, self-reporting, or other suitable measure. With further reference to embodiments of the present invention, the first measure may provide a compliance score for taking the drug. With further reference to embodiments of the present invention the first measure may provide a therapeutic window score for the drug.

### Biosensing System

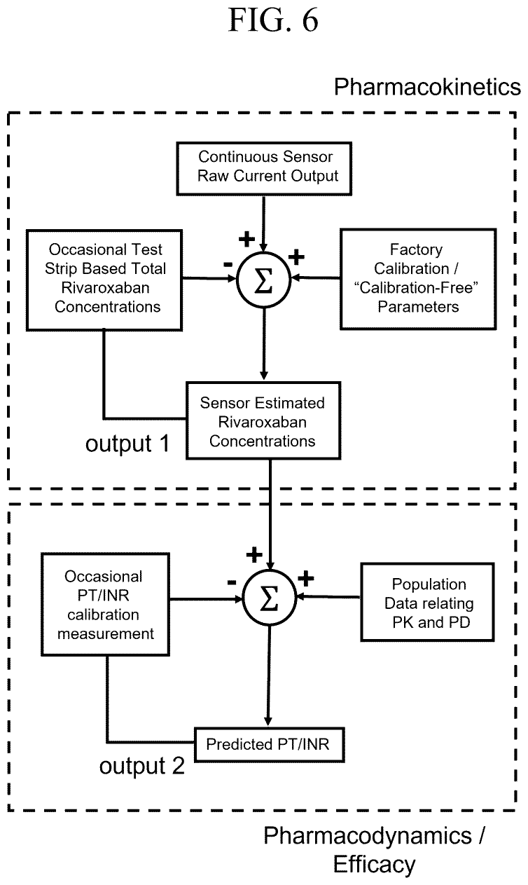

One embodiment of the present invention is a biosensing system comprising one or more devices as described herein and involving a method similar to that illustrated in FIG. 6. In one embodiment, a wearable electrochemical aptamer sensing device is worn such that the device provides a continuous or semi-continuous stream of raw current data as the sensor output. That data is then fitted to batch-calibrated, factory-calibrated, and/or “calibration-free” parameters, as is well known in the literature for electrochemical aptamer sensors, and as a result, the device is able to provide continuous or semi-continuous estimates of drug concentrations in the body. An example of such a drug is the anticoagulant drug rivaroxaban. This estimated concentration reported by the device may be corrected or updated using another biosensing device that can periodically provide discrete measures of drug concentrations. For example, a well-calibrated fingerprick test strip can be used. Alternatively, a routine blood draw that uses gold standard analytical techniques, such as HPLC/MS, can be used to provide a known concentration of rivaroxaban or another exemplary drug in the blood. This feedback loop of continuous data being updated by discrete measurements can lend itself to more accurate continuous estimates of rivaroxaban or other drug concentrations in order to provide an individual with a fully personalized pharmacokinetic profile for their current dosing regimen.

Furthermore, this continuous estimate of drug concentration/pharmacokinetic data can be used to predict a patient's individual pharmacodynamic response to a given drug. As an example, a pharmacodynamic response for rivaroxaban would be an estimate of the patient's INR or prothrombin time (PT). With regards to the exemplary method illustrated in FIG. 6, the continuous output of pharmacokinetic data could be fed into models derived from the literature that relate the pharmacokinetics of the drug to its pharmacodynamic response based on aggregate data across the population. As a result, from the sensor's continuous estimate of rivaroxaban or other drug concentration, a preliminary continuous estimate of a given patient's pharmacodynamic response can be estimated. For rivaroxaban this would result in a continuous estimate of PT/INR. This estimate can further be corrected and updated based on patient-specific data collected from either occasional use of at-home fingerprick coagulation monitors or from routine blood work done to assess PT/INR, as would be the case for the example of measuring rivaroxaban and estimating coagulation time. This occasionally collected data would lend itself to more accurate continuous estimates of the drug pharmacodynamic response based on the individual relationship between a drug's pharmacokinetics and pharmacodynamics for a given patient.

### EXAMPLES

### Example 1

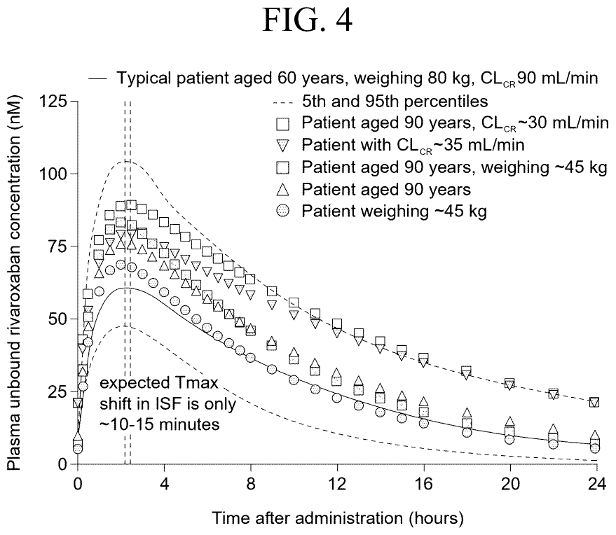

A device similar to a continuous glucose meter incorporates a gold coated wire (or in an alternative embodiment, a gold wire itself) to add an aptamer sensor for rivaroxaban. Alternative embodiments may incorporate an aptamer sensor for another blood thinner such as apixaban or warfarin. Using data from the sensor, one or more of the following measurements are conducted: (1) compliance, also known as adherence (did the patient take the medication as indicated or when levels are low enough that the next dose should be taken); (2) individual pharmacokinetic profile for the drug in the patient, for example as shown in the plots of FIG. 4; (3) coagulation time, derived directly from the drug concentration using either a pre-programmed algorithm, such as in FIG. 6, lookup table, or other suitable method using data such as that shown in FIG. 3, reported as prothrombin time, INR, other suitable score or measure that is important to a patient or doctor.

With further reference to this example, measure (3) described in the previous paragraph (coagulation time) can be even more accurate if the device is calibrated using a lab assay or at home coagulation test. The calibration can be performed in one of multiple ways. For example, the calibration can be prompted to be performed at a fixed time after dosing of the medication (such as 4 hours). For example, the calibration could be performed when the sensor reaches maximum concentration of drug in the body (call Cmax) or 2 hours after Cmax occurs, referred to at a time called Tmax which is at Tmax. For example, calibration could be performed at a time when a certain concentration is achieved, for example at 40 nM which is within the therapeutic window of the drug, to ensure calibration is optimized within the therapeutic window and not at extremes of concentrations of drug (low or high extremes such as 1 nM or 100 nM.

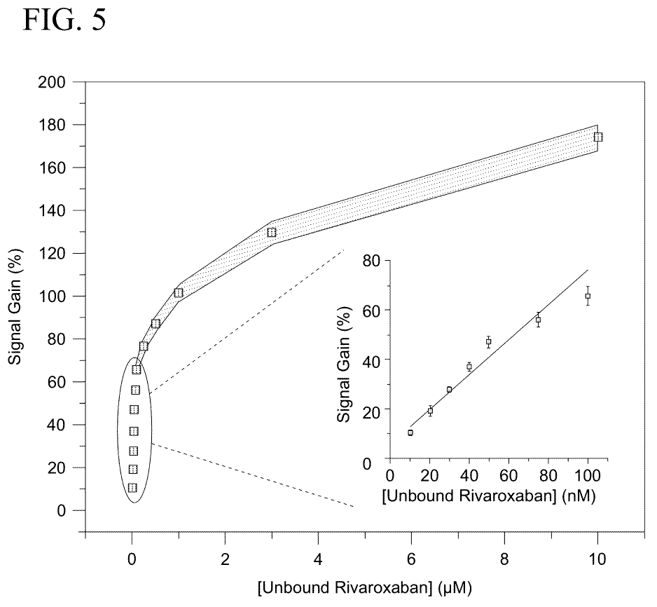

With further reference to this example, a suitable aptamer sensor is created on the gold electrode by first creating a rough, nanoporous gold surface in efforts to enhance sensor sensitivity. Briefly, such a surface is achieved electrochemically via rapid oxidation and reduction at the gold surface such that the evolution of hydrogen and oxygen gas during the process causes the gold to form nanostructures surrounding the gas bubbles as the gold is rapidly oxidized and reduced. The sensor is then formed by attaching a thiolated aptamer sequence capable of binding a target analyte of interest to the nanoporous gold surface and subsequently filling in unoccupied gold sites on the surface with an appropriate passivating molecule, such as 6-mercapto-1-hexanol or 8-mercapto-1-octanol. In one embodiment, the sensor is further protected by the addition of a hydrogel such as cross-linked polysulfobetaine or other suitable protective membranes to prevent sensor fouling and improve sensor accuracy. Results are shown for such a sensor in FIG. 5, using an aptamer distally tagged with a methylene blue redox tag with thiol binding functional group and a DNA sequence of 5 MB-GGACGACACCGCTGCGATACGGTGATACAATTGTACCGCACTGGATTGTCGT-3THIOM6SS.

SEQ. 1 is:

GGACGACACCGCTGCGATACGGTGATACAATTGTACCGCACTGGATTG
TCGT

Measurements were made in a standard electrochemical cell using an Ag/AgCl reference and a platinum counter electrode at 33 °C in bovine serum. Square wave voltammetry measurements were taken at 180 Hz with a 4 mV increment in blank serum and with subsequent additions of rivaroxaban. A signal gain of ~8% is achieved for each 10 nM of rivaroxaban across the typical therapeutic range of rivaroxaban. Therefore, if a sensor was calibrated such that a signal gain of 20% correlated with 25 nM of rivaroxaban, that same sensor could report a prothrombin time of 14 to 18 seconds, or if calibrated with a coagulation measurement report a prothrombin time of 16 seconds. If the sensor readout were to reach 100 nM, it could report to the patient a warning that they are at risk of bleeding because their concentration of drug is too high. If the sensor were to reach a drug concentration of 5 nM it could report to the patient a warning that they are at risk of thrombosis which could cause a heart attack or stroke. The same device could also give a prompt to a patient on when to take their medication or when the next dose should be taken, providing a message such as “your coagulation time is currently 25 seconds and your next dose of medicine should be taken at 8:30 PM.”

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A system for monitoring the efficacy of a drug administered to a subject comprising:
a. at least one aptamer sensor capable of providing a first measurement of the drug; and
b. a second device or sensor capable of providing a second measurement regarding the efficacy or impact of the drug.

2. The system of claim 1 wherein the second measurement is selected from the group consisting of a point of care test, a lab assay, a heart rate measurement, and a blood pressure measurement.

3. The system of claim 1 wherein the first measurement provides a compliance score for taking the drug.

4. The system of claim 1 wherein the first measurement provides a therapeutic window score for the drug.

5. The system of claim 1 wherein the first measurement is continuous and the second measurement is intermittent.

6. The system of claim 5 wherein second measurement is used to calibrate the continuous first measurement into a continuous predictive measure of the intermittent second measurements.

7. The system of claim 1 wherein the drug is an anticoagulant and the second measurement is coagulation time.

8. The system of claim 7 wherein the coagulation time measurement occurs from about an hour to two days after the drug was administered to the subject.

9. The system of claim 1 wherein the drug is predinisone and the second measurement is a level of C-reactive protein or IL-6.

10. The system of claim 1 wherein the system measures a drug and its effect, and further, wherein the effect cannot be measured with another aptamer sensor.

11. The system of claim 10 wherein the drug is an antibiotic and the effect is bacterial killing kinetics of the antibiotic afforded by area under a concentration curve.

12. The system of claim 10 wherein the drug is an antibiotic and the effect is time spent above a minimal inhibitory concentration for a given bacterial strain.

13. The system of claim 10 wherein the drug is digoxin and the effect is change in left ventricular ejection fraction time.

14. The system of claim 10 wherein the drug is theophylline and the effect is change in forced expiratory volume.

15. The system of claim 10 wherein the drug is an antiarrhythmic drug and the effect is change in electrocardiogram features.

16. The system of claim 1 wherein the second measurement includes a time-stamp and the time-stamp is aligned with time-stamps of the first measure.

17. The system of claim 1 wherein the second measurement includes a population data set taken from multiple patients taking the drug.

18. A method of reporting the pharmacodynamics of a drug in a patient, the method comprising;
a. in-vitro calibrating a sensor before use by the patient to create a calibration data set;
b. applying the sensor to the patient and measuring raw output of the sensor over a plurality of time points;
c. using the calibration data set to turn the raw output of the sensor into a quantitative measure of drug concentration in the patient;
d. taking a measurement of a pharmacodynamic effect of the drug on the patient using a point-of-care or laboratory measurement, and assigning the time of measurement of the pharmacodynamic effect to a time stamp;
e. using the measure of pharmacodynamic effect to calibrate the raw output of the sensor or the quantitative measure of the drug concentration at the time of the time stamp into a quantitative measure of pharmacodynamic effect;
f. using the measure of pharmacodynamic effect to provide a plurality of predictive quantitative pharmacodynamic effect measures over time.

19. The method of claim 18 wherein the drug is an antibiotic and the pharmacodynamic effect is bacterial killing kinetics of the antibiotic afforded by area under a concentration curve.

20. The method of claim 18 wherein the drug is an antibiotic and the pharmacodynamic effect is time spent above a minimal inhibitory concentration for a given bacterial strain.

21. The method of claim 18 wherein the drug is digoxin and the pharmacodynamic effect is change in left ventricular ejection fraction time.

22. The method of claim 18 wherein the drug is theophylline and the pharmacodynamic effect is change in forced expiratory volume.

23. The method of claim 18 wherein the drug is an antiarrhythmic drug and the pharmacodynamic effect is change in electrocardiogram features.

24. A method for providing continuous estimates of a patient's pharmacodynamic response to a drug comprising:
a. having a patient wear an electrochemical aptamer sensing device, wherein the device provides a continuous or semi-continuous stream of raw current data as output from the sensing device;
b. using the raw current data in combination with calibration parameters to calculate continuous or semi-continuous estimates of drug concentrations in the patient, wherein the calibration parameters are selected from the group consisting of batch-calibrated, factory-calibrated, “calibration-free” parameters, and combinations thereof; and
c. using the continuous or semi-continuous estimates of drug concentrations in the patient to determine a pharmacokinetic profile for the patient's current dosing regimen of the drug.

25. The method of claim 24 further comprising correcting or updating the estimates of drug concentrations by using another biosensing device that can periodically provide discrete measures of drug concentrations.

26. The method of claim 25 wherein the drug is an anticoagulant.

27. The method of claim 25 wherein the drug is rivaroxaban.
